(19) United States
(12) Patent Application Publication
(10) Pub. No.: US 2002/0091308 A1
(43) Pub. Date: Jul. 11, 2002

(54) Method and apparatus for the synchronized therapeutic treatment of a life form

(76) Inventors: Nicholas N. Kipshidze, New York, NY (US); Mikhail Falkovich, Paramus, NJ (US)

Correspondence Address: Kajane McManus, P.O. Box 344, Wonder Lake, IL, 60097, US

(21) Appl. No.: 09/757,390

(22) Filed: Jan. 9, 2001

(51) Int. Cl. A61B 5/00

(52) U.S. Cl. 600/300

(57) ABSTRACT

The method and apparatus are used in the synchronized therapeutic treatment of a life form in a manner where at least one therapeutic modality is applied to an area of the body during a period of rest or activity of a cycle thereof, application of the therapeutic modality being controlled by a preprogrammed controller in response to sensed parameters influenced by the therapeutic modality.

### BACKGROUND OF THE INVENTION

1. Field of the Invention

The invention relates to method and apparatus for synchronized therapeutic treatment of a life form. More particularly, the method and apparatus provide means by which any therapeutic treatment modality can be administered in a manner where the treatment is synchronized to appropriate parameters and a rest period or active period or active period of the cycle of an organ or area of the life form being treated with a desired modality to produce enhanced effectivity of the treatment.

2. Prior Art

Heretofore various methods and apparatus have been proposed for use in controlled application of therapeutic treatment modalities.

Exemplary embodiments may be perused in U.S. Pat. Nos. 5,496,260; 5,536,815; 5,733,310; and 5,817,021.

These prior art patents do not, however, disclose a method and apparatus for controlled therapeutic treatment which may be of any known modality or type which is synchronized to the rhythmic cycle of the organ or area being treated for maximized effectiveness of the desired modality and which is correlated to sensed parameters of the life form which are affected by the treatment modality.

It is well known that the majority of pharmaceuticals, if used frequently and in increasing doses, have adverse effects. Further, in chronically ill patients continuous usage of medication may induce tolerance, decreasing effectivity over time.

In recent years extended release tablets were developed, however they are not synchronized with cycles of body processes.

With the introduction of nanotechnology, it will be possible to implant or to introduce into the human body special platforms for local or systemic synchronized drug delivery with feedback mechanisms, treatment dosing taking place through any suitable means, such as orally, intravenously, etc.

As an example, extended release antihypertensive drugs are now prescribed for patients having high blood pressure.

With the proposed technology, special drug platforms may become available which will include sensors for monitoring the blood pressure and, if sensed blood pressure increases, an appropriate release of the drug will occur. On the other hand, if sensed pressure is normal there will be no release.

As a further example, nitroglycerin in a form capable of extended release is presently available. However, tolerance soon develops, especially when patches are used. With the proposed technology a microchip could be programmed to monitor ECG parameters and the required level of release of the drug would be provided as necessary in response to parameters sensed by a cooperating sensor.

Another example of usefulness would be for controlling release of a therapeutic agent coated onto an implanted stent or the like to prevent any occlusion thereof, as necessary.

Still further, during nighttime inactivity, the body produces excessive levels of cholesterol. Currently medication used to alleviate this problem is taken by the patient in the evening hours. With the proposed system, use of the medication can be synchronized to periods of deep sleep, even in the daytime hours of rest, by measuring levels of melatonin and other biochemical substances that are released by the body during such periods of rest.

Also, for the purpose of suppressing hunger, appetite control medication is given to the patients exclusively in the morning hours of the day. With the proposed system, the medication can be administered synchronously with the periods of hunger which are detected by measuring the biochemical parameters associated with hunger.

Such principle is also be applicable to administration of many other pharmaceuticals, as well as other treatment modalities such as various temporary and permanent implants.

And, obviously, the same technology could be beneficial in administration of simple drugs, such as aspirin, pain medication, etc.

Due to the vast area to which such technology can be applicable, the examples set forth above should not be construed as limiting.

### SUMMARY OF THE INVENTION

According to the invention there is provided apparatus for therapeutic synchronized treatment of a body comprising: a preprogrammed controller having a memory, a controllable source of a desired treatment modality, at least one sensor functionally engaged to the body for monitoring a particular parameter influenced by the treatment modality, the at least one sensor being functionally engaged to the controller for providing sensed parameter readings to the controller, the controllable source of the desired treatment modality being functionally engaged to the body and being engaged to the controller in a manner whereby the controller controls application of the treatment modality from the source, the controller analyzing the sensed parameter readings from the sensor and in response thereto, controlling the application by the source in a predetermined manner as preprogrammed into the memory of the controller.

Further according to the invention there is provided a method for accomplishing the synchronized treatment of a body comprising the steps of, programming a controller having a memory to use sensed body parameters to administer a desired therapeutic modality to the body in predetermined manner relative to the sensed body parameters, engaging at least one controllable source of a desired therapeutic modality to the controller for controlling the application of the modality and to the body, engaging at least one sensor to the controller and to the body, the sensor monitoring a particular body parameter influenced by the at least one therapeutic modality, the controller analyzing input from the sensor and, in response thereto, controlling the application of the at least one treatment modality from the source to the body in the predetermined manner programmed into the controller.

### BRIEF DESCRIPTION OF THE DRAWINGS

FIG. 1 is a block diagram of generic, exemplary embodiment of a synchronized therapy apparatus made in accordance with the teachings of the present invention.

FIG. 2 is an exemplary time line showing timing of application of one treatment modality for a particular condition using the method of the present invention, which may be accomplished using the apparatus of FIG. 1.

### DESCRIPTION OF THE PREFERRED EMBODIMENTS

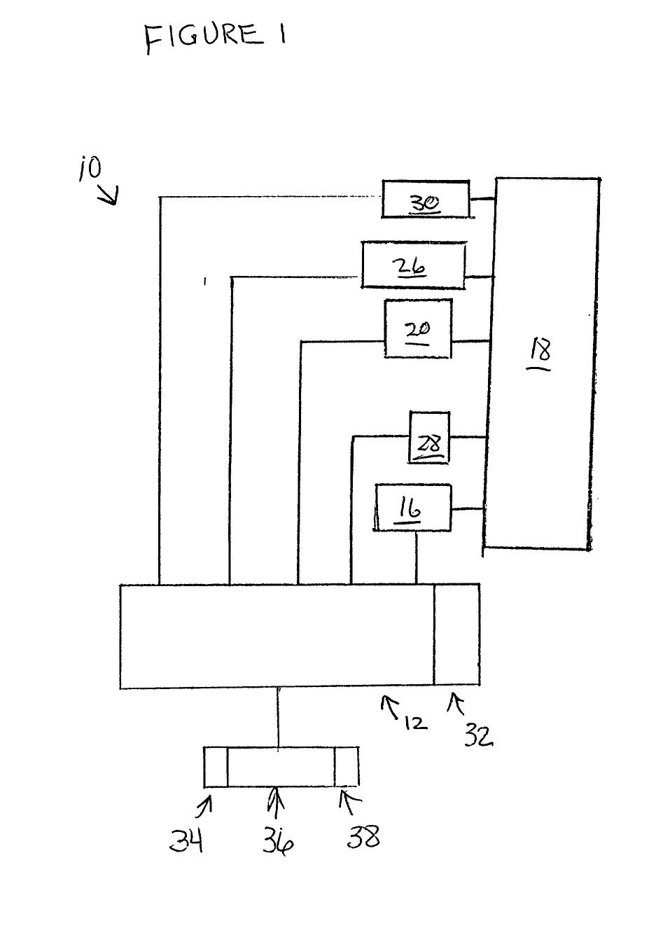

Referring now to the drawings in greater detail, there is illustrated in FIG. 1 a schematic diagram of a generic embodiment of a synchronized therapy apparatus made in accordance with the teachings of the present invention which will be referred to hereinafter by the reference numeral 10, and which is used in administration of the method of the present invention.

The apparatus 10 comprises a central synchronizing control module 12 which forms the heart of the apparatus 10.

With respect to the methodology, it will be understood that most bodily processes have a particular cyclic rhythm and sequence. For example, each smooth muscle has a known particular frequency and rhythm. It has been found that application of a desired treatment modality in a manner which is synchronized to the particular cycle of the organ or area being treated will provide the greatest effectivity of the modality of treatment when the treatment is administered during the rest period common to each cycle.

The monitoring of such cyclic parameters to generate maximized effectivity of a therapeutic treatment modality by application of the modality during the rest period of the cycle has not heretofore been proposed.

The present invention revolves around monitoring exactly such cyclic parameters, natural or induced, and provides maximization of therapeutic effectivity by application of the treatment modality during the rest period, regardless of the type of therapeutic treatment modality used.

Viewing FIG. 1 it will be understood that at least one therapeutic treatment modality 16 may be applied to a particular area of a life form 18 to be treated in a manner synchronized to the cycle of the area such that treatment is applied only during a sensed rest period of the cycle of the area.

For provision of such synchronized treatment, at least one appropriate sensor 20 which senses a parameter affected by the treatment modality 16 in use, is functionally connected to the life form 18 for monitoring the parameter. Concurrently, a secondary sensor 26 which senses a different secondary parameter affected by the treatment modality in use may also be operatively engaged to the life form 18 if it is desired to determine when a cyclic rest period for the area begins, such point being defined as ΔT.

It will also be understood that more than one treatment modality may be applied to the life form 18, concurrently with the first modality 16.

Application of such necessary secondary modality 28 is also accommodated by the apparatus 10 and method.

Still further, although most areas of a life form 18 are found to have a particular cyclic rhythm and sequence, there are certain areas where such does not exist, and it may be desired to induce a rhythmic cycle, such as during treatment of a skeletal muscle, for example.

To this end a cycling mechanism 30 of any known functional type, may be utilized to generate a desired cycle.

It will be obvious also that the controller 12 must include a memory 32 for storing and accessing parameter and treatment variables for a particular life form 18 being treated.

Still further, because parameters for each life form 18 are different, an input device 34 and a display 36 are provided for selective programming of the controller 12 and visually monitoring apparatus 10 function, respectively.

Since the memory 32 will also be preprogrammed with limits for the apparatus 10, an alarm 38 will be provided to indicate that limits have been violated and human intervention is required.

Considering the treatment modalities 16 and 28, it is proposed that each may comprise any one of at least a therapeutic ultrasound transducer, a therapeutic x-ray tube, laser fiber optics, a pulse generator, a gated source of medication, an energy emitter, etc. Thus, any therapeutic modality the application of which is controllable, is accommodated, and the above examples should not be construed as limiting.

Considering the cycling mechanism 30, it is proposed that any known stimulator suitable for stimulating the particular area being treated which is controllable in the application thereof, such as electrostimulation or mechanical cycling, may be used.

With respect to the primary and/or secondary sensors 20 and 26, respectively, such may comprise any suitable sensing form for the particular condition being treated such as at least a pulse oximeter, diagnostic ultrasound, an electroencephalograph, an electrocardiograph, an electromyelograph, an sphygmomanometer, or any other type of sensor required for monitoring the particular parameter being dealt with.

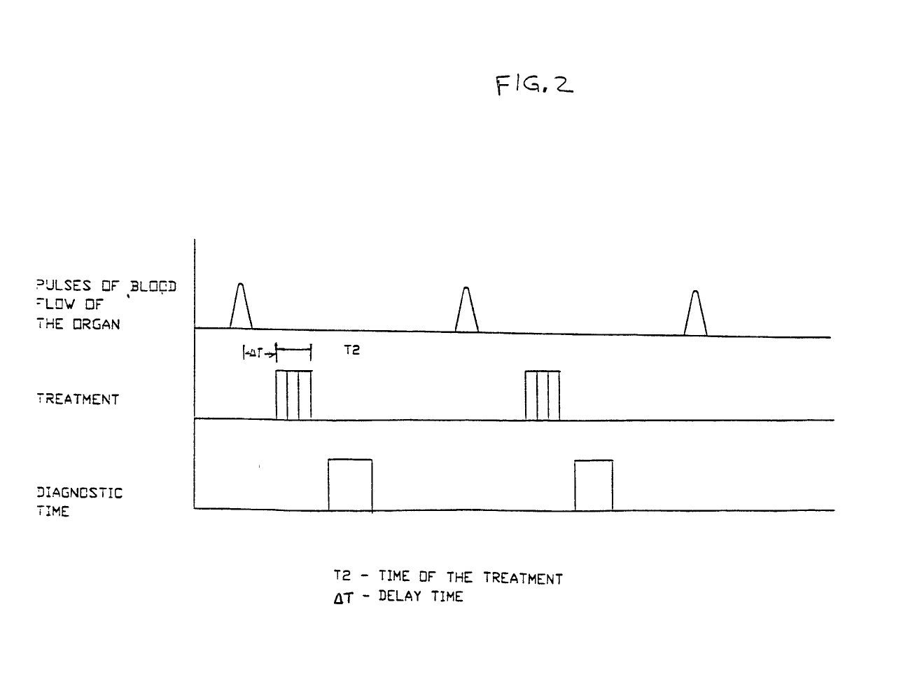

Although the concepts of the method and apparatus 10 for accomplishing the method should be clear to those skilled in the art from the above discussion, an example using one particular treatment modality 16 is presented in FIG. 2, in the form of a time line for further clarification.

In this example, an area of the life form 18 to be treated is defined as a particular organ (not shown) of the life form 18. Also, in this example, let the desired treatment modality 16 comprise therapeutic ultrasound. For provision of synchronized treatment using the method and apparatus 10, for this therapeutic embodiment, diagnostic ultrasound 26, is operative for use in determining the period of rest for the organ.

The determined delay ΔT is used as the basis for determining the timing for application of therapeutic ultrasound 16, labelled T2, to assure that the treatment modality 16 is applied in a manner synchronized to the cyclic rhythm of the organ and, by taking into account the delay ΔT, the treatment modality is applied during the period of rest of the organ for increased effectivity of treatment.

It will be understood that using available nanotechnology, it has become possible to identify resting periods of areas to be treated.

It has been found that application of a therapeutic modality during a rest or an active period of the organ will provide maximized effectivity of the treatment based on the condition being treated. Sensing of the effect of the applied treatment modality 16 is proposed to be accomplished during the rest period, for adjustment of timing, delay, duration, amplitude, etc., of the next cycle of treatment based on sensed parameters produced by the previous application of the treatment modality 16.

With respect to the few bodily processes that are asynchronous, such as, for example, activity of a pectoral muscle,continuous synchronicity can be certificially produced.

In this respect although there is no continuous synchronicity in the activity of such a muscle, once continuous and repetitive activity has begun, the sequence of events is identical for each activation.

In such a case, activation can be induced as necessary, by stimulating the muscle with the cycling mechanism 30 which, for a muscle, would be by application of electrical pulse, in known manner.

Once the activation cycle has begun, with the cyclic sequence of events being known, the activation sequence can be easily monitored, such that, once contraction has taken place and the muscle begins its relaxation phase, treatment would then be applied with effectivity of treatment also being monitored during the relaxation phase, as described above.

It will be further understood that the monitoring, as well as modality of treatment, may take any of various known forms which are suitable, as based on the particular area and particular condition being treated.

For example, in the treatment of diabetes, one might monitor blood sugar as well as blood flow through the pancreas. Obviously, one would not monitor blood CO₂ content in this case, while CO₂ content in blood would be suitable for monitoring when dealing with treatment of ischemic heart disease.

Further, as iterated above, treatment modalities could run the gamut of any which are suitable for treating a particular condition in a controllable manner. In this respect, when dealing with diabetes, for example, insulin could be administered through an IV drip, or when dealing with a muscle, a relaxant could be administered, or, in treating other disorders, ultrasound or radiation could be applied, etc.

Control of administration of a required treatment modality 16, in response to continually sensed and input conditional parameters is accomplished in the apparatus 10 by the central processing unit or controller 12 which is preprogrammed to follow a predetermined plan which is patient specific, as in any therapy.

The programming would also allow for manual override by a physician, when necessary, to accommodate potential extenuating circumstances which may exist. For example, if a patient were extremely obese, requiring administration of a level of treatment modality 16 above a predefined upper limit, such upper limit could be overridden or reset by the physician based on the particular needs for the particular patient. As a specific example, if a patient requiring therapeutic ultrasound treatment of an organ is obese, in order to meet a required threshold of applied energy for eliciting a therapeutic response, it may be necessary to increase the level of power output while decreasing time of application, in known manner.

It will be understood that certain conditions will be more responsive to treatment during an active period of a cycle. In the system defined, such condition will be treated only during active periods. Accordingly the system and method will be understood to be useful in the rest or active periods of a cycle, and are never applied across the entire cycle.

Further, the controller 12 would be programmed to activate the alarm 38 if any predefined limits were exceeded, with application of the particular modality 16 being stopped until modifications to parameters brought them back within limits.

As described above, the method and apparatus of the present invention provide a number of advantages, some of which have been described above and others of which are inherent in the invention. Also, modifications may be proposed without departing from the teachings herein. Accordingly the scope of the invention is only to be limited as necessitated by the accompanying claims.

1. Apparatus for therapeutic synchronized treatment of a body comprising:
a preprogrammed controller having a memory;
a controllable source of a desired treatment modality;
at least one sensor functionally engaged to the body for monitoring a particular parameter influenced by the treatment modality;
the at least one sensor being functionally engaged to the controller for providing sensed parameter readings to the controller;
the controllable source of the desired treatment modality being functionally engaged to the body and being engaged to the controller in a manner whereby the controller controls application of the treatment modality by the source;
the controller analyzing the sensed parameter readings from the sensor and in response thereto, controlling application by the source in a predetermined manner as preprogrammed into the memory of the controller.

2. The apparatus of claim 1 further incorporating a cycling device for synchronous stimulation of an asynchronous area of the body, the cycling device being functionally engaged to the body and the controller.

3. The apparatus of claim 1 wherein the controller includes a display for visually presenting sensor and treatment parameters.

4. The apparatus of claim 1 wherein the controller further includes an alarm for indicating any parameter outside programmed limits.

5. The apparatus of claim 1 wherein the controller further includes structure for manually overriding programmed parameters.

6. The apparatus of claim 1 where a primary sensor can be any one of at least a pulse oximeter with probe, diagnostic ultrasound, EEG, EMG, ECG and NIBP module.

7. The apparatus of claim 1 wherein a secondary sensor which cooperates with a primary sensor can be any other one of at least a pulse oximeter with probe, diagnostic ultrasound, EEG, EMG, ECG and NIBP module.

8. The apparatus of claim 1 wherein the at least one modality of treatment comprises any one of a therapeutic ultrasound, therapeutic radiation, laser, magnetic pulse generator, energy emitter, gated medication source and any other controllable treatment modality.

9. The apparatus of claim 8 wherein a second cooperating modality of treatment comprises any other one of a therapeutic ultrasound, therapeutic radiation, laser, magnetic pulse generator, energy emitter, gated medication source and any other controllable treatment modality.

10. The apparatus of claim 1 wherein the cycling device comprises one of a mechanical cycler and an electrostimulator.

11. The apparatus of claim 1 wherein the controllable treatment modality is applied during a period of rest of each cycle of the body area being. treated.

12. The apparatus of claim 1 wherein the controllable treatment modality is applied during a period of activity of each cycle of the body area being treated.

13. The apparatus of claim 1 wherein the at least one sensor is activated after application of the treatment modality during a period of rest of each cycle of the body area being treated.

14. The apparatus of claim 1 wherein the at least one sensor is activated after application of the treatment modality during a period of activity of each cycle of the body area being treated.

15. A method for accomplishing the synchronized treatment of a body comprising the steps of:
programming a controller having a memory to use sensed body parameters to administer a desired therapeutic modality to the body in predetermined manner relative to the sensed body parameters;
engaging at least one controllable source of a desired therapeutic modality to the controller for controlling application of the modality and to the body;
engaging at least one sensor to the controller and to the body, the sensor monitoring a particular body parameter influenced by the at least one therapeutic modality;
the controller analyzing input from the sensor and, in response thereto, controlling the application of the at least one treatment modality from the source to the body in the predetermined manner programmed into the controller.

16. The method of claim 15 wherein a cycling device for synchronous stimulation of an asynchronous area of the body is engaged to and between the body area and the controller, with the controller controlling operation of the device.

17. The method of claim 15 wherein a display is provided to visually present sensor and treatment parameters.

18. The method of claim 15 wherein an alarm is engaged to and activated by the controller when any sensed parameter is outside predetermined limits.

19. The method of claim 15 wherein a manual override is provided in the controller.

20. The method of claim 15 wherein the treatment modality is only applied to the body during a certain period of rest of a cycle of the area being treated.

21. The method of claim 15 wherein the treatment modality is only applied to the body during a certain period of activity of a cycle of the area being treated.

22. The method of claim 15 wherein the sensor is activated to sense the desired parameter after application of the treatment modality during a period of rest of a cycle of the area being treated.

23. The method of claim 15 wherein the sensor is activated to sense the desired parameter after application of the treatment modality during a period of activity of a cycle of the area being treated.
